(19) United States
(12) Patent Application Publication
(10) Pub. No.: US 2008/0176293 A1
(43) Pub. Date: Jul. 24, 2008

(54) RNA-Dependent RNA Polymerase, Methods And Kits For The Amplification And/Or Labelling Of RNA

(76) Inventors: Jacques Rohayem, Dresden (DE); Wolfram Rudolph, Dresden (DE); Katrin Jaeger, Dresden (DE); Enno Jacobs, Dresden (DE)

Correspondence Address: ST. ONGE STEWARD JOHNSTON & REENS, LLC, 986 BEDFORD STREET, STAMFORD, CT, 06905-5619, US

(21) Appl. No.: 12/019,206

(22) Filed: Jan. 24, 2008

Related U.S. Application Data
(63) Continuation of application No. PCT/DE2006/001326, filed on Jul. 25, 2006.

(30) Foreign Application Priority Data
Jul. 25, 2005 (DE) 10 2005 036 085.8
Feb. 13, 2006 (DE) 10 2006 008 219.2

(51) Int. Cl. C12P 19/34; C12N 9/10

(52) U.S. Cl. 435/91.2; 435/193

(57) ABSTRACT

The invention relates to an RNA-dependent RNA-polymerase, to methods and kits for marking and/or amplifying in a primary dependent or independent manner a ribonucleic acid (RNA), in particular a viral, eucaryontic, procaryontic and double-stranded ribonucleic acid (RNA) for the biological and medical use thereof. Said RNA-dependent RNA-polymerase comprises a right-hand conformation and the amino acid sequence the following sequence sections: a. XXDYS b. GXPSG c. YGDD d. XXYGL e. XXXXFLXRXX having the following significances: D: aspartate, Y: tyrosine, S: serine, G: glycine, P: proline, L: leucine, F: phenylalanine, R: arginine, X: any amino acid. The inventive amplifying method is particularly suitable for microarray engineering, siRNA production and for diagnosticating viral infections by detecting viral RNA in patient samples. The inventive marking method is particularly suitable for purifying RNA by means of affinity bonds and for marking RNA used in molecular biology methods for characterising the function and/or structure of a viral, eucaryontic, procaryontic and double-stranded ribonucleic acid (RNA).

### CROSS-REFERENCE TO RELATED APPLICATIONS

The present application is a continuation of pending International patent application PCT/DE2006/001326 filed on Jul. 25, 2006 which designates the United States and claims priority from German patent application Nos. 10 2005 036 085.8 filed on Jul. 25, 2005 and 10 2006 008 219.2 filed Feb. 13, 2006, the content of which is incorporated herein by reference.

### FIELD OF THE INVENTION

The invention relates to an RNA-dependent RNA polymerase as well as to methods and kits for labelling and/or to the primer-dependent and -independent amplification of ribonucleic acid (RNA), in particular viral, eukaryotic and prokaryotic and double-stranded ribonucleic acid (RNA) for application in biotechnology and medicine. The amplification method according to the present invention is particularly applicable to microarray technology, preparation of siRNA and diagnosis of viral infections through the detection of viral RNA in patient material. The labelling method according to the present invention is particularly suited for the purification of RNA by affinity binding as well as for the labelling of RNA for the application in molecular biological procedures used in the characterisation of the function and/or structure of viral, eukaryotic and prokaryotic and double-stranded ribonucleic acid (RNA).

### BACKGROUND OF THE INVENTION

RNA is an important component of the eukaryotic and the prokaryotic cell. It is involved in several vital functions: the transcription, i.e. the transliteration of information in the genome (deoxyribonucleic acid, DNA), the transmission of this information from the cell nucleus to the cytoplasm (mRNA), and the translation (i.e. the transformation of the transcribed information into amino acids or proteins, respectively). RNA also forms the so-called transfer-RNA which is an important component for translation. RNA is an important component of the ribosomes as well. These are structural entities of the cytoplasm on which the translation of the information into proteins takes place.

The significance of RNA in the field of biotechnology has increased in the past decades. New developments such as microarray technology, miRNA (micro-RNA) or siRNA-technology (small interfering RNA) have evolved, the relevance of which in the field of basic research as well as in applied research is increasing. All these technologies are based on the synthetic generation of RNA through amplification in vitro.

So far, the preparation of siRNA in vitro is carried out by chemical synthesis or with the aid of DNA-dependent RNA polymerases such as the T7 RNA polymerase. For this purpose, two complementary DNA-strands are synthesised by PCR employing a primer additionally containing the sequence for the T7 promoter. After transcription of the DNA with the aid of the T7 RNA polymerase the resulting complementary RNA strands are hybridised and digested with RNAse 1.

In the medical field RNA amplification plays an important role in the detection of viral infections in patient material. 80% of all viral infections worldwide are caused by RNA-viruses, i.e. viral pathogens having a genome consisting of RNA. Important pathogens within this group of viruses are amongst others the human immunodeficiency virus (HIV), hepatitis C virus (HCV), hepatitis A virus, influenza virus (influenza A, B and C) but also avian pest virus, the recently newly identified SARS virus, polio virus, measles virus, mumps virus, rubella virus and combined diarrhea and vomiting viruses (rotavirus, sapovirus and norovirus).

So far, the detection of RNA in patient material is carried out in four steps. After recovering the RNA from the material under examination the subsequent steps follow:
1. Transcription of the RNA in copy-DNA (cDNA) through RNA-dependent DNA polymerases, the so-called transcriptases. A primer is required for this step. Primers are oligonucleotides being specific for the sequence to be amplified. Primers and the RNA-dependent DNA polymerase enable the targeted amplification of genetic material, though in the form of DNA;
2. Amplification of the cDNA through polymerase chain reaction (PCR);
3. Purification of the PCR product;
4. Sequencing the PCR product.

In the case of RNA preparation, for example for expression detection for preparation of siRNA, an additional transcription of the PCR-product into RNA through DNA-dependent RNA polymerases, for example T7 polymerase, is carried out.

The accomplishment of these steps takes, as a rule, 18 to 24 hours. Likewise, certain “know-how” is required which is not accessible for all research institutions.

A further method for the amplification of RNA is provided by the replication through RNA-dependent RNA polymerases such as for example the RNA polymerase of bacteriophage Qβ (also denoted as Qβ replicase), the RNA polymerase of polio virus or of hepatitis C virus.

The genome of bacteriophage Qβ consists of a single stranded (+)-RNA which is replicated by the RNA-dependent RNA polymerase. The Qβ replicase produces a (−)-strand RNA copy of the (+)-strand, which can also function as a template like the (+)-strands. Thus, an exponential amplification can be attained. The Qβ system is already in use for the detection of analytes, such as nucleic acids (Chu et al. (1986), Nucleic Acids Research 14, 5591-5603; Lizardi et al. (1988), Biotechnology 6, 1187-1202).

However, these RNA-dependent RNA polymerases require certain RNA-sequences for initiation of RNA-replication. Furthermore, they also depend on certain helper proteins for the amplification of RNA. For this reason, these RNA-dependent RNA polymerases are incapable of amplifying heterologous RNA.

The labeling of RNA at the 3′-terminus according to the prior art is only feasible of by means of ATP through the use of E. coli poly(A) transferase. Methods for labelling RNA with CTP, UTP and GTP are unknown.

Therefore, the technical problem underlying the invention is the provision of an RNA-dependent RNA polymerase as well as methods and kits for labelling and/or amplifying viral, eukaryotic and prokaryotic single and double stranded ribonucleic acids (RNA), which are more efficient and faster.

### SUMMARY OF THE INVENTION

According to the present invention, the solution to the problem is provided by an RNA-dependent RNA polymerase (in the following also denoted as RdRP or 3D^{pol}) for the amplification and/or labelling of RNA, wherein the RNA-dependent RNA polymerase belongs to the viruses of the family of Caliciviridae and having a “right hand conformation” and wherein the amino acid sequence of the RNA-dependent RNA polymerase comprises the following sequence motifs:

a. XXDYS

b. GXPSG

c. YGDD

d. XXYGL

e. XXXXFLXRXX

having the following meanings:
D: aspartate
Y: tyrosine
S: serine
G: glycine
P: proline
L: leucine
F: phenylanaline
R: arginine
X: any amino acid.

A so-called “right hand conformation” means that the tertiary structure (conformation) of the protein has a folding according to a right hand with finger, palm and thumb.

The sequence motif “XXDYS” is the so-called A-motif. The A-motif accounts for the discrimination between ribonucleosides and deoxyribonucleosides.

The sequence motif “GXPSG” is the so-called B-motif. The B-motif is conserved between all representatives of the Caliciviridae family.

The sequence motif “YGDD” is the so-called C-motif. The C-motif represents the active centre of the enzyme. This motif plays an important role in the coordination of the metal ions.

The sequence motif “XXYGL” is the so-called D-motif. The D-motif is a feature of the template-dependent polymerases.

The sequence motif “XXXXFLXRXX” is the so-called E-motif. The E-motif is a feature of the RNA-dependent RNA polymerases and is not present in DNA-dependent polymerases.

The RNA-dependent RNA polymerase of the present invention has the following functions:
Mg²⁺— or Mn²⁺-dependent RNA-dependent RNA polymerase activity, no DNA-dependency;
template-dependency;
primer-dependent RNA synthesis with homopolymeric RNA (poly(A)-RNA, poly(C)-RNA, poly(G)-RNA or poly(U)-RNA, respectively) as template in the presence of corresponding primers (i.e. oligo(U)-RNA, oligo(G)-RNA, oligo(C)-RNA or oligo(A)-RNA, respectively);
primer-independent RNA-synthesis with poly(C)-RNA as template in the presence of elevated GTP concentrations;
primer-dependent or primer-independent RNA synthesis with heteropolymeric RNA as template;
terminal transferase activity with preference for UTP and CTP leading to the labelling of the 3′-end of a single-stranded RNA template.

The primer-dependent amplification of RNA occurs only in the presence of a sequence-specific primer. The amplification is sequence-dependent and occurs through incorporation of AMP, GMP, CMP or UMP.

The primer-independent amplification of the RNA occurs in the absence of a sequence specific primer. The amplification is sequence dependent and occurs through incorporation of AMP, GMP, CMP or UMP.

Due to the terminal transferase activity multiple nucleotides of one kind (for example ATP, UTP, CTP or GTP) are added to the 3′-end of an RNA strand independent of the sequence of said RNA strand.

The RNA-dependent polymerase of the present invention is surprisingly capable of employing heterologous viral, eukaryotic and prokaryotic RNA as a template for the amplification reaction in vitro. Both positive-stranded and negative-stranded, single-stranded and double-stranded RNA can be utilised for amplification.

Preferably, the RNA-dependent RNA polymerase according to the present invention is a RNA-dependent RNA polymerase of a virus of the Caliciviridae family. The genome of the Caliciviridae consists of a polyadenylated (+)-stranded single RNA strand. In vivo the RNA-dependent RNA polymerase of the calicivirus transcribes the genomic calicivirus RNA into a (−)-stranded antisense-RNA (aRNA) in the course of replication. In doing so, an RNA-aRNA-hybrid is generated. Subsequently, the (−)-stranded aRNA serves as the template for the synthesis of new (+)-stranded genomic virus RNA.

In further preferred embodiments of the invention the RNA-dependent RNA polymerase is an RNA-dependent RNA polymerase of a human and/or non-human pathogenic calicivirus. Especially preferred is an RNA-dependent RNA polymerase of a norovirus, sapovirus, vesivirus or a lagovirus, for example the RNA-dependent RNA polymerase of the novovirus strain HuCV/NL/Dresden174/1997/GE or of the sapovirus strain pJG-Sap01 or of the vesivirus-strain FCV/Dresden/2006/GE.

According to especially preferred embodiments of the invention the RNA-dependent RNA polymerase is a protein having a amino acid sequence according to SEQ ID NO 1, SEQ ID NO 2 or SEQ ID NO 3.

SEQ ID NO 1 is the amino acid sequence of a norovirus-RdRP.
SEQ ID NO 2 is the amino acid sequence of a sapovirus-RdRP.
SEQ ID NO 3 is the amino acid sequence of a vesivirus —RdRP.

SEQ ID NO 1:
MGGDSKGTYCGAPILGPGSAPKLSTKTKFWRSSTTPLPPGTYEPAYLGGK
DPRVKGGPSLQQVMRDQLKPFTEPRGKPPKFSVLEAAKKTIINVLEQTID
PPEKNSFTQACASLDKTTSSGHPHHMRKNDCWNGESFTGKLADQASKANL
MFEGGKNMTPVYTGALKDELVKTDKIYGKIKKRLLWGSDLATMIRCARAF
GGLMDELKAHCVTLPIRVGMNMNEDGPIIFRRHSHYKYHYDADYSRWDST
QQRAVLAAALEIMVKFSSEPHLAQVVAEDLLSPSVVDVGDFKISINEGLP
SGVPCTSQWNSIAHWLLTLCALSEVTNLSFDIIQANSLFSFYGDDEIVST
DIKLDPEKLTAKLKEYGLKPTRPDKTEGPLVISEDLNGLTFLRRTVTRDP
AGWFGLKEQSSILRQMYWTRGPNHEDPSETMIPHSQRPIQLMSLLGEAAL
HGPAFYSKISKLVIAELKEGGMDFYVPRQHPMFRWMRFSDLSTWEGDRNL
APSFVNEDGVEVDKLAAALE
SEQ ID NO 2:
MKDEFQWKGLPVVKSGLDVGGMPTGTRYHRSPAWPEEQPGETHAPAPFGA
GDKRYTFSQTEMLVNGLKPYTEPTAGVPPQLLSRAVTHVRSYIETIIGTH
RSPVLTYHQACELLERTTSCGPFVQGLKGDYWDEEQQQYTGVLANHLEQA
WDKANKGIAPRNAYKLALKDELRPIEKNKAGKRRLLWGCDAATTLIATAA
PKAVATRLQVVTPMTPVAVGINMDSVQMQVMNDSLKGGVLYCLDYSKWDS
TQNPAVTAASLAILERFAEPHPIVSCAIEALSSPAEGYVNDIKFVTRGGL
PSGMPFTSVVNSINHMIYVAAAILQAYESHNVPYTGNVPQVETVHTYGDD
CMYSVCPATASIFHAVLANLTSYGLKPTAADKSDAIKPTNTPVFLKRTFT
QTPHGVRALLDITSITRQFYWLKANRTSDFESPPAFDRQARSAQLENALA
YASQHGPVVPDTVRQIAIKTAQGEGLVLVNTNYDQALATYNAWFIGGTVP
DPVGHTEGTHKIVFEME
SEQ ID NO 3:
MKVTTQKYDVTKPDISYKGLICKQLDEIRVIPKGTRLHVSPAHTDDYDEC
SHQPASLGSGDPRCPKSLTAIVVDSLKPYCEKTDGPPHDILHRVQRMLID
HLSGFVPMNISSEPSMLAAFHKLNHDTSCGPYLGGRKKDHMIGGHPDKPL
LDLLSSKWKLATQGIGLPHEYTIGLKDELRPVEKVQEGKRRMIWGCDVGV
ATVCAAAFKGVSDAITANHQYGPVQVGINMDGPSVEALYQRIRSAAKVFA
VDYSKWDSTQSPRVSAASIDILRYFSDRSPIVDSAANTLKSPPIAIFNGV
AVKVTSGLPSGMPLTSVINSLNHCLYVGCAILQSLESRNIFVTWNLFSTF
DMMTYGDDGVYMFPMMFASVSDQIFANLTAYGLKPTRVDKSVGAIEPIDP
ESVVFLKRTITRTPHGIRGLLDRGSIIRQFYYIKGENSDDWKTPPKTIDP
TSRGQQLWNACLYASQHGPEFYNKVYRLAEKAVEYEELHFEPPSYHSALE
HYNNQFNGVDTRSDQIDASVMTDLHCDVFELVE

The RNA-dependent RNA polymerases of the norovirus, the sapovirus and the vesivirus are recombinantly producible through cloning of the nucleic acid coding for the norovirus-, sapovirus- or vesivirus-RdRP, respectively, into a suitable expression vector, for example pET-28 (Novagen). The expression vector carrying the gene sequence coding for the virus RdRP is introduced into a suitable host organism for expression. The host organism is preferably selected from prokaryotic, preferably Escherichia coli, or eukaryotic host organisms, preferably Sacharomyces cerevisae, or insect cells (preferably Sf9 cells), which have been infected with recombinant baculoviruses, which are host organisms commonly used for the protein expression. This host organism contains at least one expression vector having a gene sequence coding for the virus RdRP.

According to preferred embodiments the norovirus, sapovirus or vesivirus calicivirus RdRP is fused at its N- or C-terminus to a protein sequence which facilitates the purification of the RdRP fusion proteins after expression in a host organism. Preferably this sequence is a so-called histidine marker which consists of a sequence of at least 6 consecutive histidines (H is or H). Such a histidine marker allows the purification of the protein by affinity chromatography over a nickel or cobalt column in a known manner.

Examples of embodiments of the RNA-dependent RNA polymerase fused to a histidine marker are the proteins having a amino acid sequence according to SEQ ID NO 4, SEQ ID NO 5 or SEQ ID NO 6.

SEQ ID NO 4 is the amino acid sequence of a novovirus-RdRP having a histidine marker (His-tag).

SEQ ID NO 5 is the amino acid sequence of sapovirus-RdRP having a histidine marker (His-tag).

SEQ ID NO 6 is the amino acid sequence of a vesivirus-RdRP having a histidine marker (His-tag).

SEQ ID NO 4:
MGGDSKGTYCGAPILGPGSAPKLSTKTKFWRSSTTPLPPGTYEPAYLGGK
DPRVKGGPSLQQVMRDQLKPFTEPRGKPPKPSVLEAAKKTIINVLEQTID
PPEDWSFTQACASLDKTTSSGHPHHMRKNDCWNGESFTGLKADQASKANL
MFEGGKNMTPVYTGALKDELVKTDKIYGKIKKRLLWGSDLATMIRCARAF
GGLMDELKAHCVTLPIRVGMNMNEDGPIIFERHSRYKYHYDADYSRWDST
QQRAVLAAALEIMVKFSSEPHLAQVVEADLLSPSVVDVGDFKISINEGLP
SGVPCTSQWNSIAHWLLTTCALSEVTNLSPDIIQANSLPSFYGDDEIVST
DIKLDPEKLTAKLKEYGLKPTRPDKTEGPLVISEDLNGLTFLRRTVTRDP
AGWFGLKEQSSILRQMYWTRGPNHEDPSETMIPHSQRPIQLMSLLGEAAL
HGPAFYSKISKLVIAELKEGGMDPYVPRQEPMFRWMRFSDLSTWEGDRNL
APSFVNEDGVEVDKLAAALEHHHHHH
SEQ ID NO 5:
MKDEFQWKGLPVVKSGLDVGGMPTGTRYHRSPAWPEEQPGETHAPAPFGA
GDKRYTFSQTEMLVNGLKPYTEPTAGVPPQLLSRAVTHVRSYIETIIGTH
RSPVLTYHQACELLERTTSCGPFVQGLKGDYWDEEQQQYTGVLANHLEQA
WDKANKGIAPRNAYKLALKDELRPIEKNKAGKRRLLWGCDAATTLIATAA
FKAVATRLQVVTPMTPVAVGINMDSVQMQVMNDSLKGGVLYCLDYSKWDS
TQNPAVTAASLAILERFAEPHPIVSCAIEALSSPAEGYVNDIKFVTRGGL
PSGMPFTSVVNSINHMIYVAAAILQAYESHNVPYTGNVFQVETVHTYGDD
CMYSVCPATASIFHAVLANLTSYGLKPTAADKSKAIKPTNTPVFLKRTPT
QTPHGVRALLDITSITRQFYWLKANRTSDPSSPPAFDRQARSAQLENALA
YASQHGPVVFDTVRQIAIKTAQGEGLVLVNTNYDQALATYNAWFIGGTVP
DPVGHTEGTHKIVFEMEHHHHHH
SEQ ID NO 6:
MKVTTQKYDVTKPDISYKGLICKQLDEIRVIFKGTRLHVSPAHTDDYDEC
SHQPASLGSGDPRCPKSLTAIVVDSLKPYCEKTDGPPHDILHRVQRMLID
HLSGFVPMNISSEPSMLAAFHKLNHDTSCGPYLGGRKKDHMIGGEPDKPL
LDLLSSKWKLATQGIGLPHEYTIGLKDELRPVEKVQEGKRRMIWGCDVGV
ATVCAAAFKGVSDAITANHQYGFVQVGINMDGPSVEALYQRIRSAAKVFA
VDYSKWDSTQSPRVSAASIDILRYFSDRSPIVDSAANTLKSPPIAIPNGV
AVKVTSGLPSGMPLTSVINSLNHCLYVGCAILQSLESRNIPVTMNLPSTP
DMMTYGDDGVYMFPMMFASVSDQIFANLTAYGLKPTRVDKSVGAIEPIDP
ESVVPLKRTITRTPHGIRGLLDRGSIIRQFYYIKGENSDDWKTPPKTIDP
TSRGQQLWNACLYASQHGPRFYNKVYRLAEKAVEYEELHFEPPSYHSALE
HYNNQFNGVDTRSDQIDASVMTDLHCVDFEVLEHHHHHH

Part of the invention forms also the use of an RNA-dependent RNA polymerase according to the present invention for the amplification and/or labelling or marking of RNA.

Also part of the invention is a method for the amplification of RNA by means of an RNA-dependent RNA polymerase of the present invention comprising the steps of:
a. attachment or annealing, respectively, of the RNA-dependent RNA polymerase to the RNA template in the presence or absence of an oligonucleotide primer, for example an oligo-RNA-primer or oligo-DNA-primer;
b. transcription of the RNA-template into antisense RNA by the RNA-dependent RNA polymerase;
c. separation of the RNA/antisense RNA duplex into single RNA strands;
   as well as a kit for carrying out the method for the amplification of RNA, which comprises
      a. an RNA-dependent RNA polymerase according to the present invention
      b. a suitable reaction buffer
      c. NTPs
      d. optionally, RNase inhibitor
      e. optionally, stop solution.

Preferably, in the method according to the invention, the RNA template is used in amounts of 1 μg to 4 μg per 50 μl reaction volume. The concentration of the ribonucleotides ATP, CTP, GTP and UTP (NTPs) used is preferably between 0.1 μmol/l and 1 μmol/l, more preferably 0.4 μmol/l. In the method for the amplification of RNA according to the present invention, it is also possible to employ NTP analogues such as fluorescently labelled NTPs, biotin-labelled NTPs or radioactively labelled NTPs such as [P³²]-labelled NTPs. The concentration of the RdRP according to the invention is preferably between 1 μmol/l and 3 μmol/l.

According to an especially preferred embodiment, the kit contains
a. 150 μmol/l recumbently produced norovirus-RdRP according to SEQ ID NO 1 or SEQ ID NO 4 and/or sapovirus-RdRP according to SEQ ID NO 2 or SEQ ID NO 5 and/or vesivirus-RdRP according the SEQ ID NO 3 or SEQ ID NO 6
b. as buffer: 50 mmol/l HEPES, pH 8.0, 3 mmol/1 magnesium acetate or manganese chloride (MnCl₂), 4 mM DTT
c. 10 mmol/l ATP, 10 mmol/l CTP, 10 mmol/l GTP, 10 mmol/l UTP
d. RNase-inhibitor
e. as stop solution: 4 mol/l ammonium acetate, 100 mmol/l EDTA

The method is preferably carried out at a pH value of between 6.5 and 8.5 and at a temperature of between 20° C. and 40° C. A preferred embodiment of the method for RNA amplification is carried out at 30° C. with norovirus-RdRP or at 37° C. with sapovirus-RdRP under the following buffer conditions: 50 mmol/l HEPES, pH 8.0, 3 mmol/1 magnesium acetate or manganese chloride, 4 mmol/l DTT.

According to a preferred embodiment of the invention the separation of the RNA/antisense-RNA-duplexes into single RNA strands occurs through heat denaturation, chemical denaturation and/or enzymatically, for example by an enzyme having the ability of separating double-stranded RNA into single-stranded RNA such as, for example, a helicase.

Due to the strand separation a single-stranded template is present again, and a further round of amplification can be induced.

By employing an enzyme being capable of separating double-stranded RNA into single-stranded RNA, the reaction can be maintained isothermically.

Part of the invention forms a corresponding kit for the amplification of RNA which, in addition to the kit according the invention, contains an enzyme, for example a helicase, having the ability to separate double-stranded RNA into single-stranded RNA.

An embodiment of the invention is a method for the primer-dependent amplification of RNA, wherein at least one primer hybridising to a section of the RNA template is employed and wherein subsequently to the annealing of the RNA-dependent RNA polymerase according to the present invention the primer is elongated by the RNA-dependent RNA polymerase according to the sequence of the RNA template.

As a primer, preferably an RNA primer or a DNA primer, for example having a length of 20 to 25 bases, preferably at a concentration of 0.1 to 1 μmol/l, is used. As an RNA template, for example viral, prokaryotic and eukaryotic RNA is applicable.

The procedure of the primer-dependent RNA amplification of a single-stranded RNA template by use of an RNA-primer is schematically represented in FIG. 1.

It is preferred to use poly-U-RNA, poly-A-RNA, poly-C-RNA or poly-G-RNA primers as primer for polyadenylated RNA, polyuridylated, polyguanylated RNA or polycytidylated RNA, respectively. By means of the poly-U-RNA primer a specific amplification of the total cellular mRNA is feasible. For this purpose, it is preferred to use a poly-U-primer having a length of from 20 to 24 bases. The amplified cellular mRNA can be subsequently analyzed with the aid of so-called microarray methods.

The method according to the present invention for the sequence-specific RNA amplification is also advantageously used for the detection of viral RNA in patient material.

For this purpose, total cellular RNA of the patient's material is recovered and viral RNA contained in the patient material is amplified by means of an RNA-primer hybridising specifically with a specific section of viral RNA.

As the patient material, for example liquor, blood, plasma or body fluids can be used.

The method is also suitable for the detection of RNA viruses having a poly-A-tail at the 3′-end of the genome, DNA-viruses and viral mRNA transcripts.

Also part of the invention is a kit for carrying out the method for primer-dependent amplification of RNA, comprising

a. an RNA-dependent RNA polymerase of the invention
b. a suitable reaction buffer
c. NTPs
d. optionally, RNase inhibitor
e. optionally, stop solution
f. primer.

According to a preferred embodiment the kit contains
a. 150 μmol/l recombinantly produced norovirus-RdRP according to SEQ ID NO 1 or SEQ ID NO 4 and/or sapovirus-RdRP according SEQ ID NO 2 or SEQ ID NO 5 and/or vesivirus-RdRP according to SEQ ID NO 3 or SEQ ID NO 6
b. as buffer: 50 mmol/l HEPES, pH 8.0, 3 mmol/1 magnesium acetate or manganese chloride (MnCl₂), 4 mM DTT
c. 10 mmol/l ATP, 10 mmol/l CTP, 10 mmol/l GTP, 10 mmol/l UTP
d. RNase-inhibitor
e. as stop solution: 4 mol/l ammonium acetate, 100 mmol/l EDTA
f. 0.1 to 3 μM primer (homopolymeric or heteropolymeric RNA- or DNA-oligonucleotide having a length of from 15 to 20 bases)

Also part of the invention is a method for the primer-independent amplification of RNA wherein no primer hybridising with a section of the RNA template is used and elongated by the RNA-dependent RNA polymerase according to the sequence of the RNA template, as well as a kit for the primer-independent amplification of RNA which comprises

a. an RNA-dependent RNA polymerase of the invention
b. a suitable reaction buffer
c. NTPs
d. optionally, RNase inhibitor
e. optionally, stop solution.

In this embodiment of the method according to the present invention, the annealing of the RNA-dependent RNA polymerase to the RNA template is primer-independent. The procedure of the sequence-independent RNA amplification of a single-stranded RNA template in the absence of an RNA primer is schematically depicted in FIG. 2.

A further embodiment of the invention is a method for the primer-independent amplification of poly(C)-RNA characterized in that no primer hybridising with a section of the RNA template is employed, that GTP, preferably 50 μM, is used as the single nucleotide and elongated by the RNA-dependent RNA polymerase according to the sequence of the RNA template.

The schematic process of the sequence-independent RNA synthesis based on a poly(C)-RNA in the presence of 50 μM GTP is shown in FIG. 4.

Possible applications of the method according to the present invention for the amplification of RNA are numerous. On the one hand, the direct detection of viral genetic material in patient material is made feasible. For this purpose, total cellular RNA is recovered, and any RNA sequence is specifically amplified by use of an RNA primer. The detection of the viral nucleic acid then occurs through hybridisation to specific probes. Another application relates to the unspecific (RNA primer-independent) amplification of the RNA, which can make feasible the identification of hitherto unidentified viruses and new viral variants, respectively.

A further application relates to microarray technology This aims to differentially detect cellular expression. This occurs through detection of so-called cellular transcripts, i.e. the mRNA. Based on a cellular mixture, the invention allows the specific amplification of mRNA using a poly(U)-oligonucleotide in order to subsequently yield the detection by hybridisation on microarrays.

A further field of application of the invention represents the production of siRNA in vitro, which is thitherto carried out with the aid of T7 polymerase. For this purpose,
1. two complementary DNA double-strands are synthesised by means of PCR using a T7 promoter primer;
2. after transcription of the DNA with the aid of T7 polymerase, both resulting complementary RNA strands are hybridised;
3. then, both strands are digested by RNAse I.

According to the prior art, these steps commonly take 24 to 48 hours and also afford the required know-how. On the contrary, the invention allows for the direct, efficient and simple preparation of double-stranded RNA starting from a RNA sequence without the necessity of a PCR step, of in vitro transcription and of the hybridisation of the RNA (which potentially proceeds suboptimal).

Also part of the invention is a method for labelling or marking, respectively, RNA using an RNA-dependent RNA polymerase according to the present invention, comprising the steps of

a. annealing of the RNA-dependent RNA polymerase to the RNA to be labelled;

b. adding at least one nucleotide to the 3′-end of the RNA to be labelled;

as well as a kit for carrying out the inventive method for the labelling of RNA, which comprises

a. an RNA-dependent RNA polymerase according to the present invention

b. a suitable reaction buffer

c. CTP or UTP or ATP or GTP

d. optionally, RNase inhibitor

e. optionally, stop solution.

The process of labelling the 3′-end of the RNA template is schematically depicted in FIG. 3.

According to a particularly preferred embodiment the kit contains
150 μmol/l recombinantly produced norovirus-RdRP according to SEQ ID NO 1 or SEQ ID NO 4 and/or sapovirus-RdRP according SEQ ID NO 2 or SEQ ID NO 5 and/or vesivirus-RdRP according to SEQ ID NO 3 or SEQ ID NO 6
as buffer: 50 mmol/l HEPES, pH 8.0, 3 mmol/1 magnesium acetate or manganese chloride (MnCl₂), 4 mM DTT
10 mmol/l ATP, 10 mmol/l CTP, 10 mmol/l GTP, 10 mmol/l UTP RNase-inhibitor
as stop solution: 4 mol/l ammonium acetate, 100 mmol/l EDTA.

### BRIEF DESCRIPTION OF THE DRAWINGS

The present invention is further illustrated with reference to the following figures and examples. The figures show:

FIG. 1: flow chart of the sequence-dependent RNA amplification;

FIG. 2: flow chart of the sequence-independent RNA amplification;

FIG. 3: flow chart of the labelling of the 3′-end of the RNA template;

FIG. 4: flow chart of the sequence-independent RNA synthesis starting from a poly(C)-RNA in the presence of 50 μM GTP;

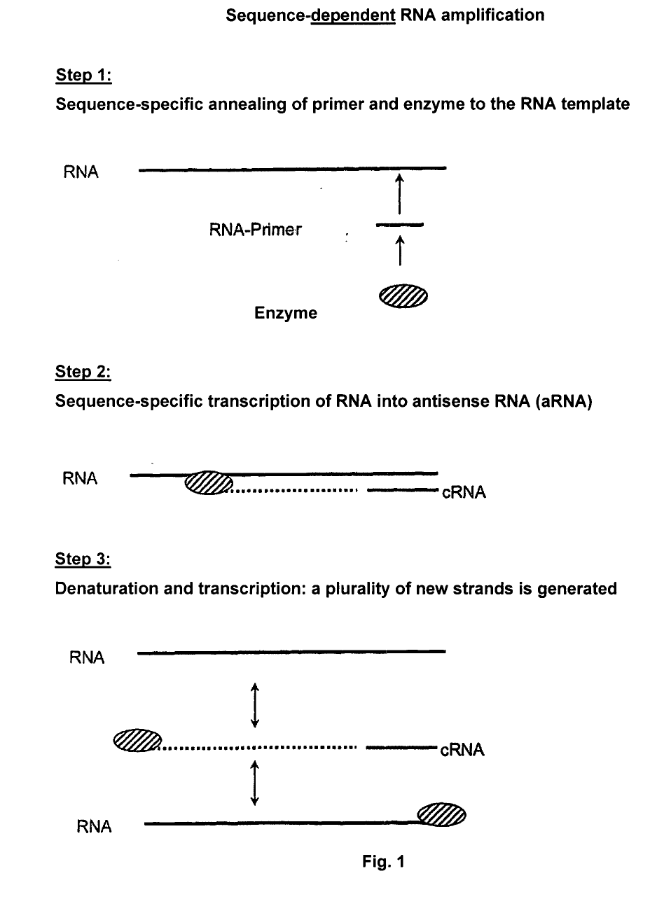

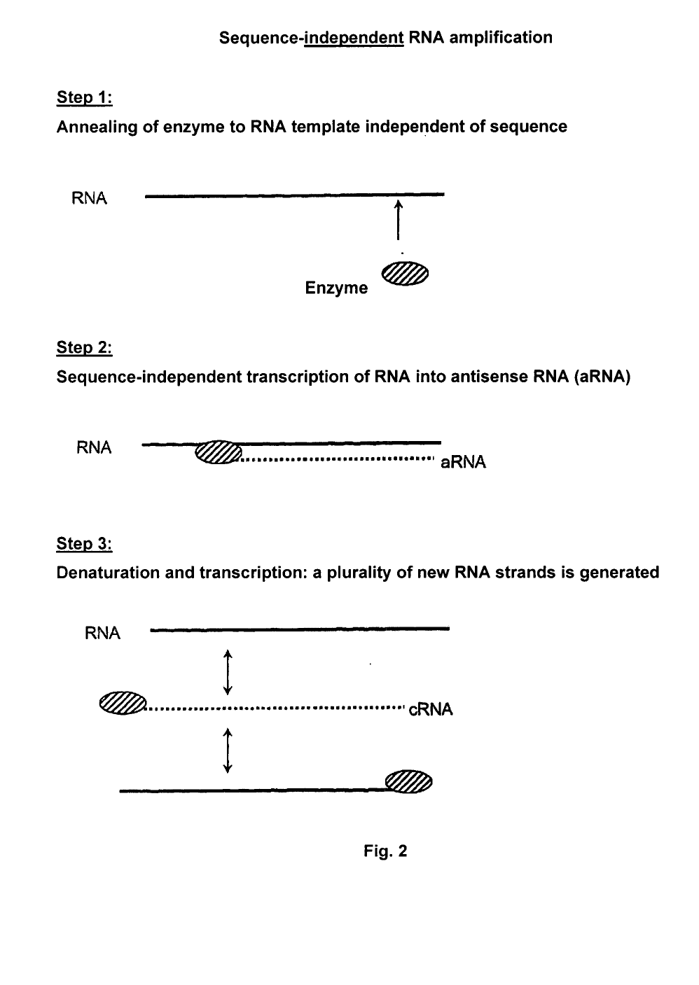

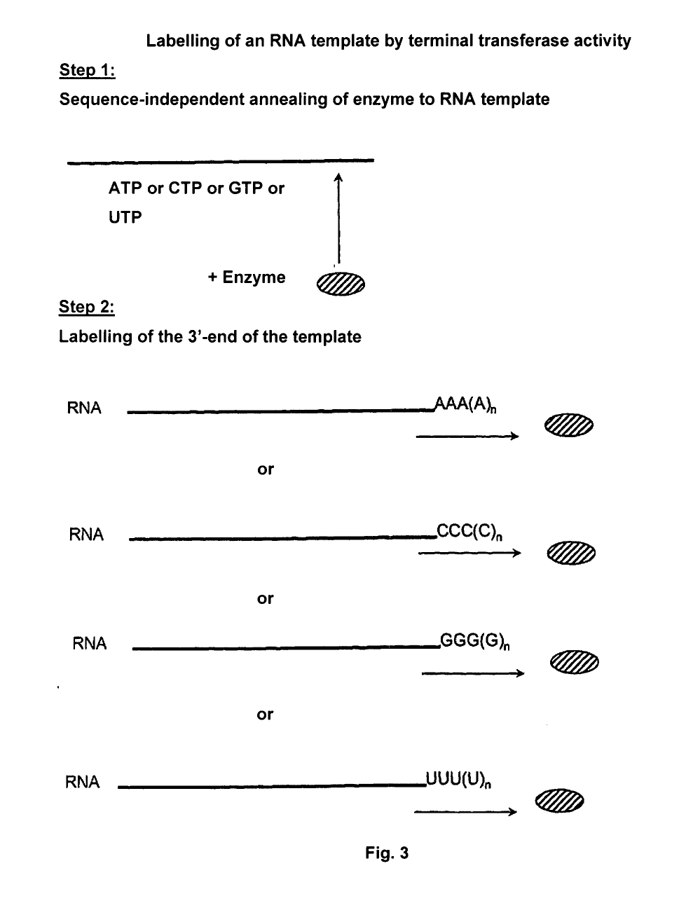

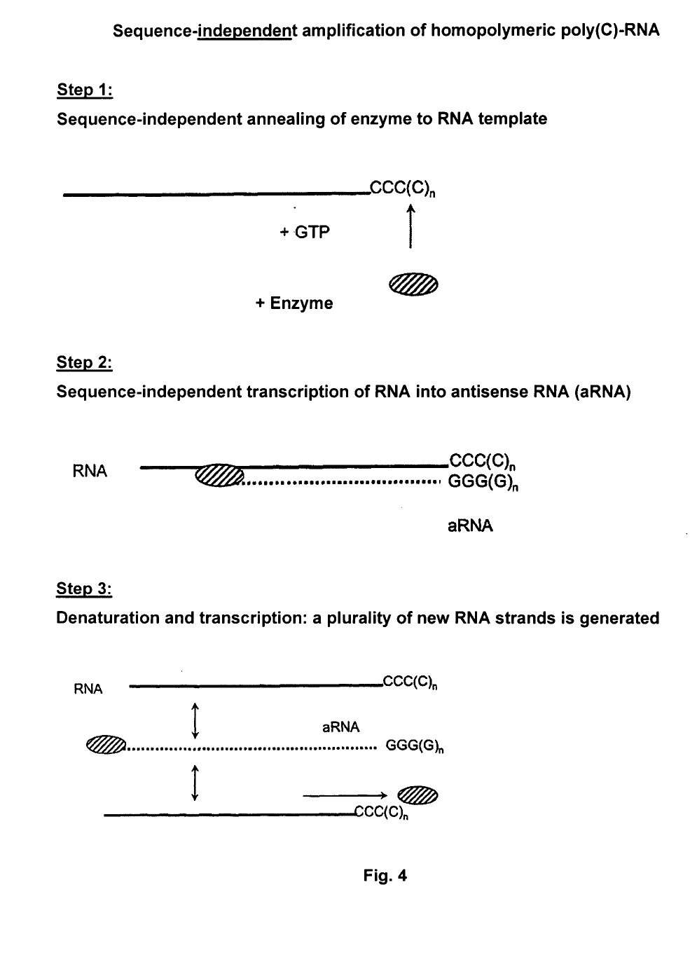

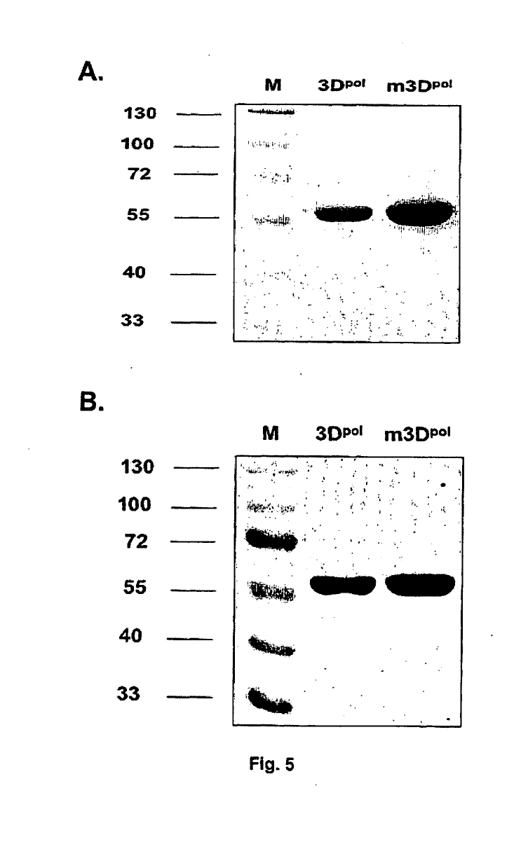

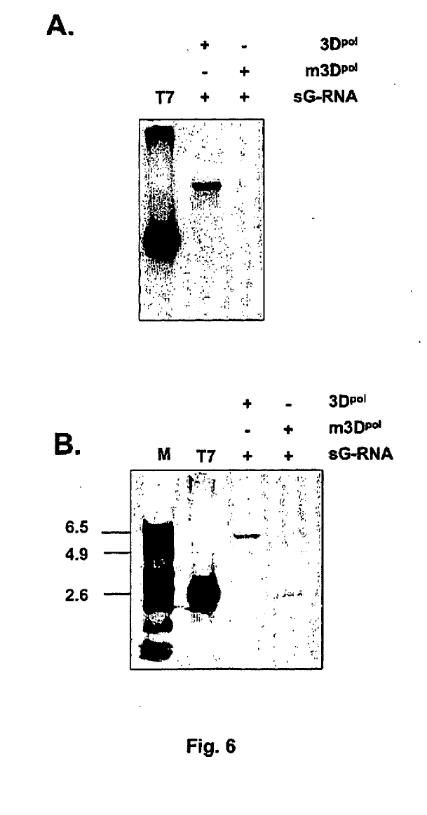

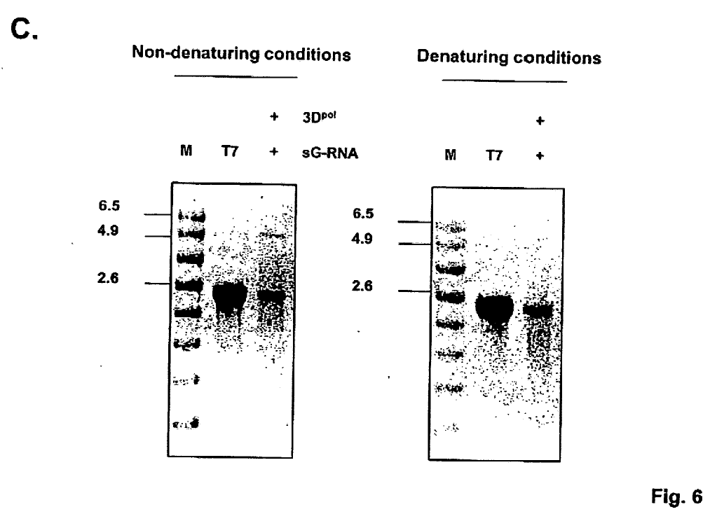

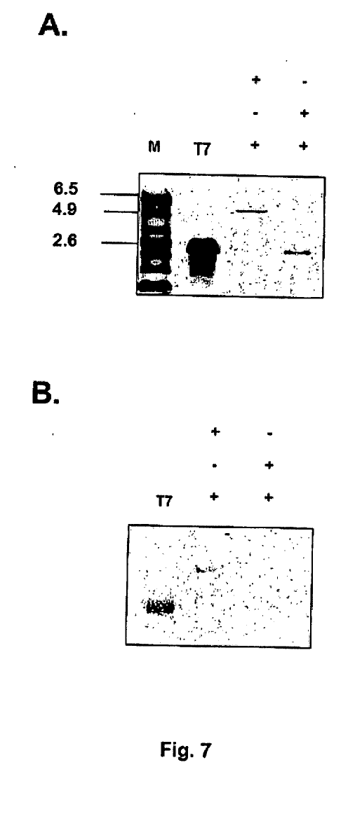

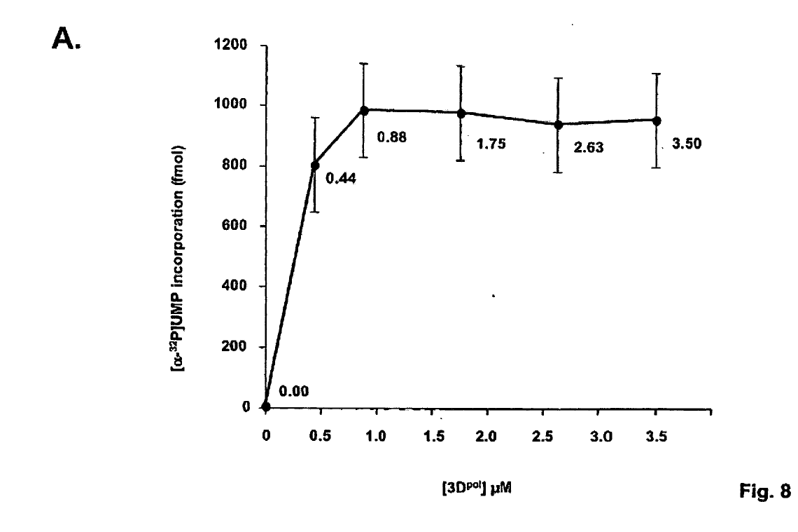

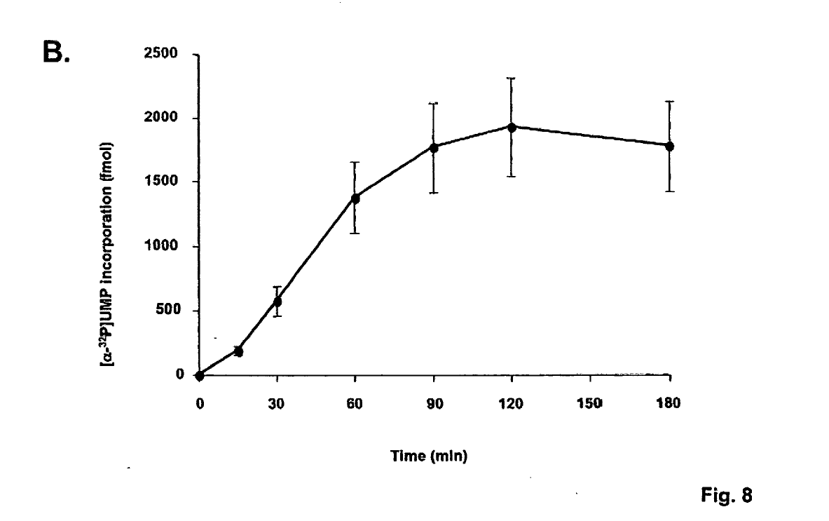

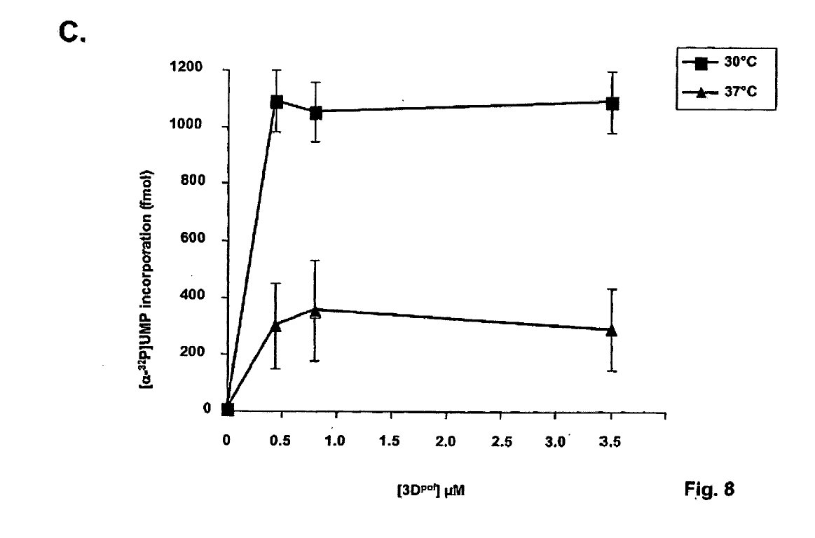

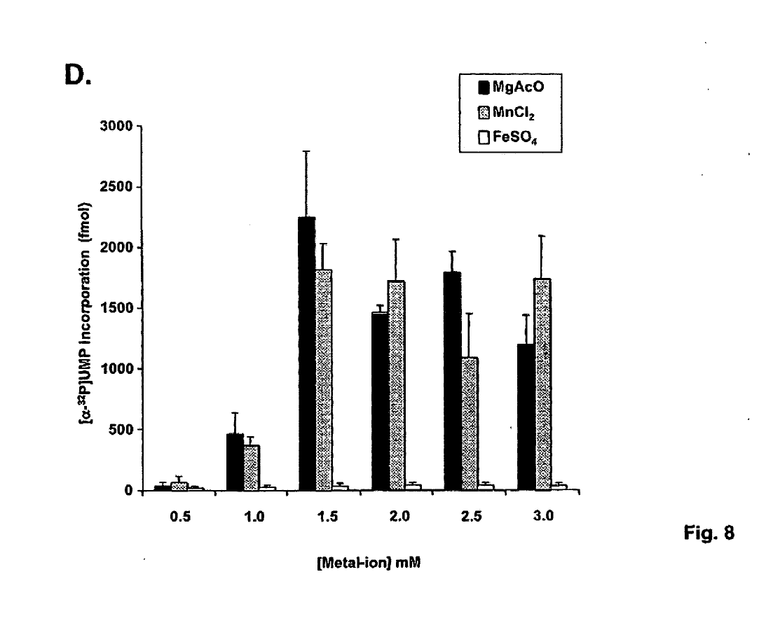

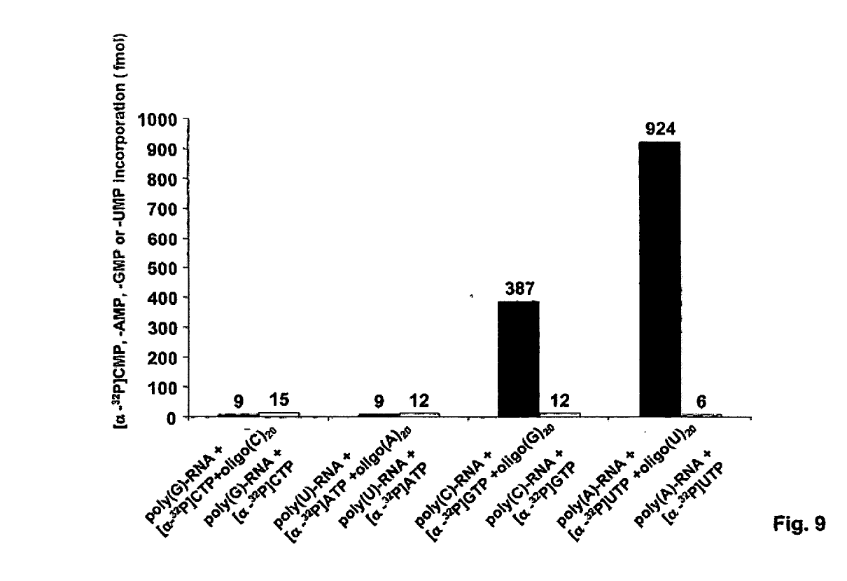

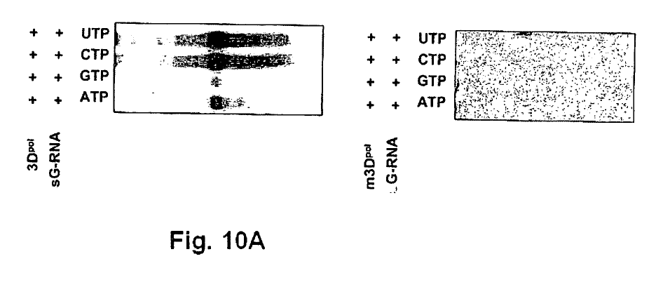

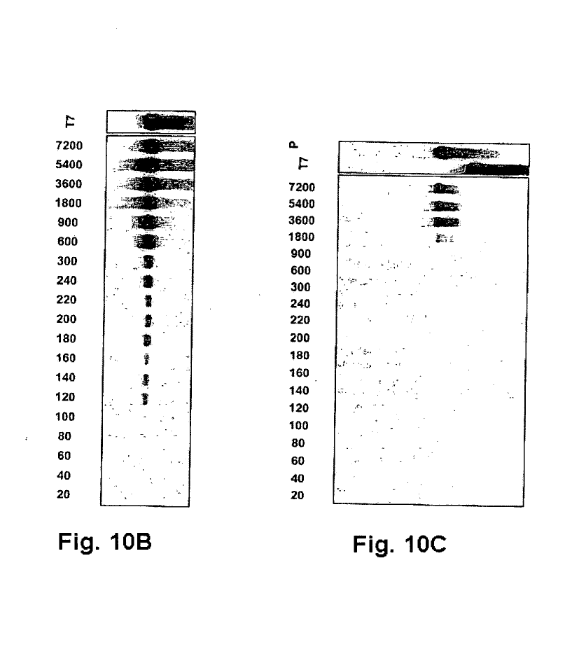

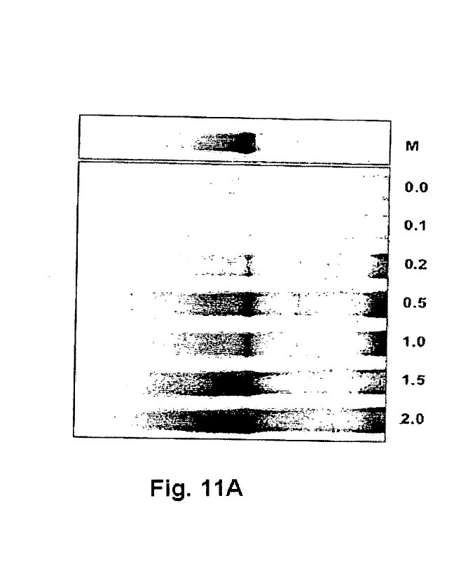

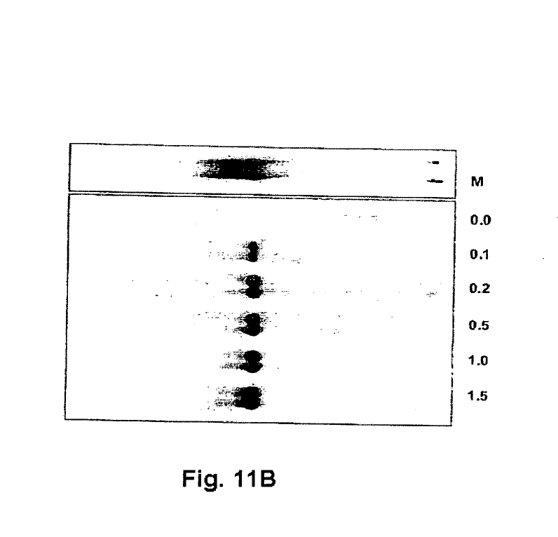

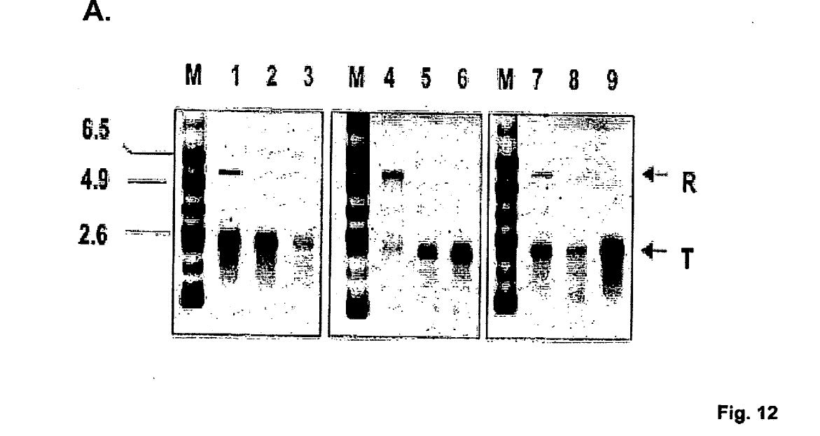

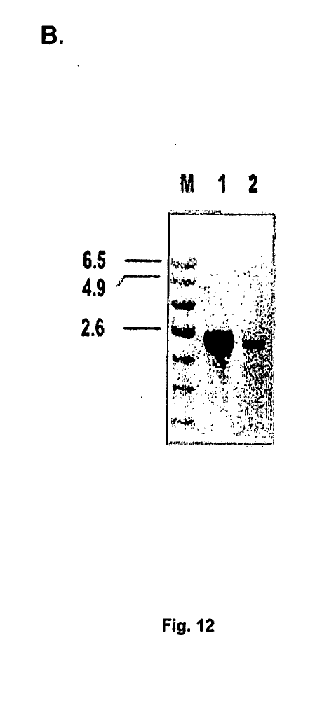

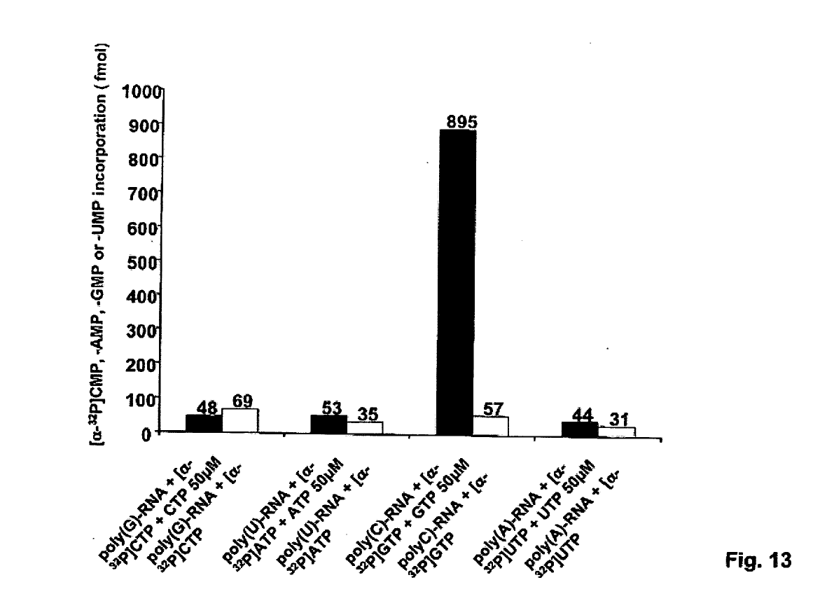

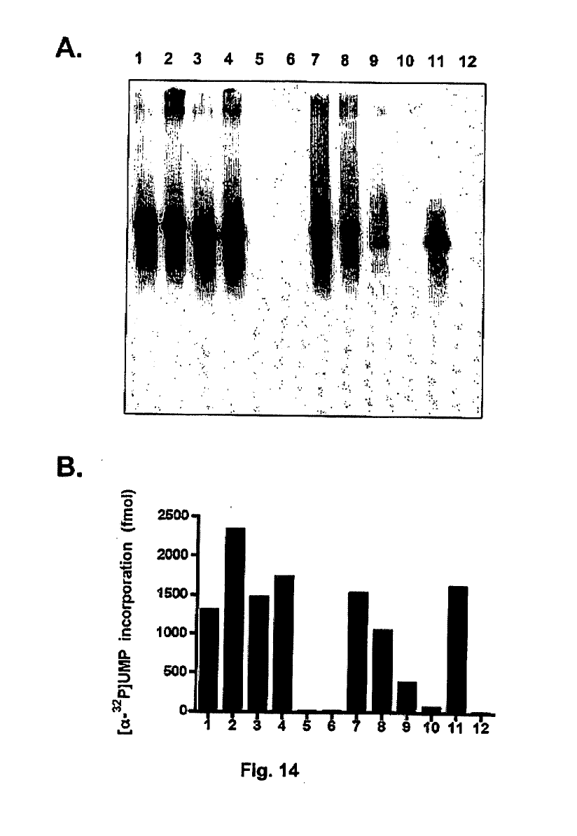

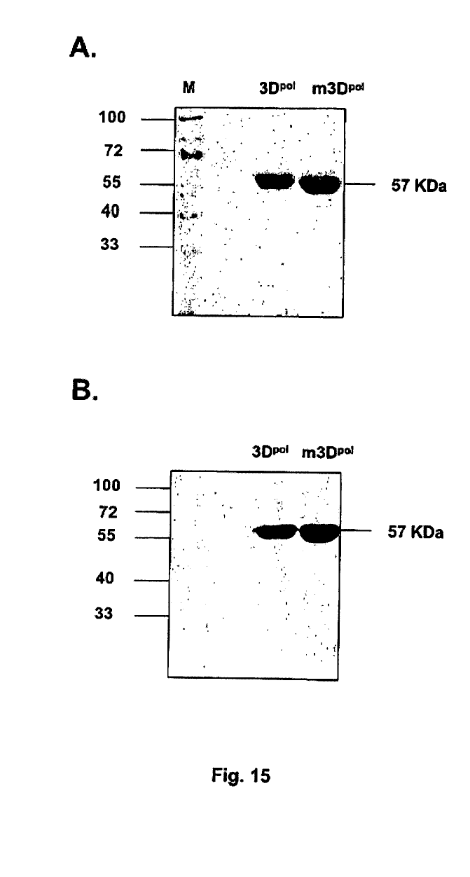

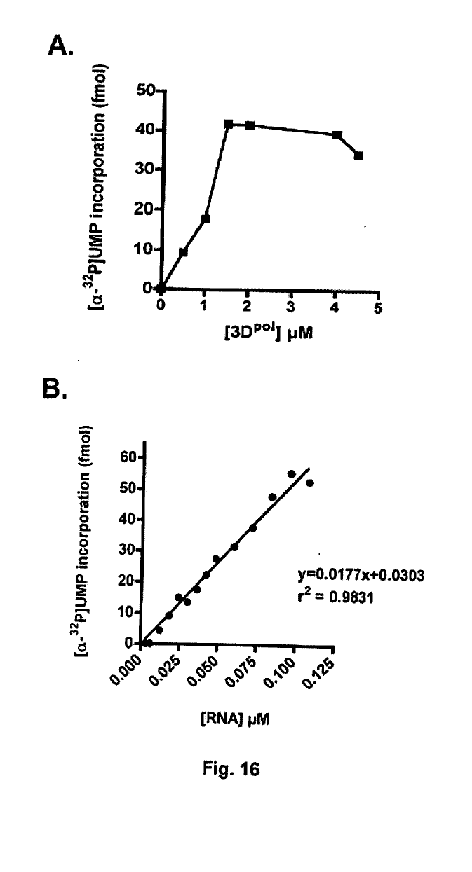

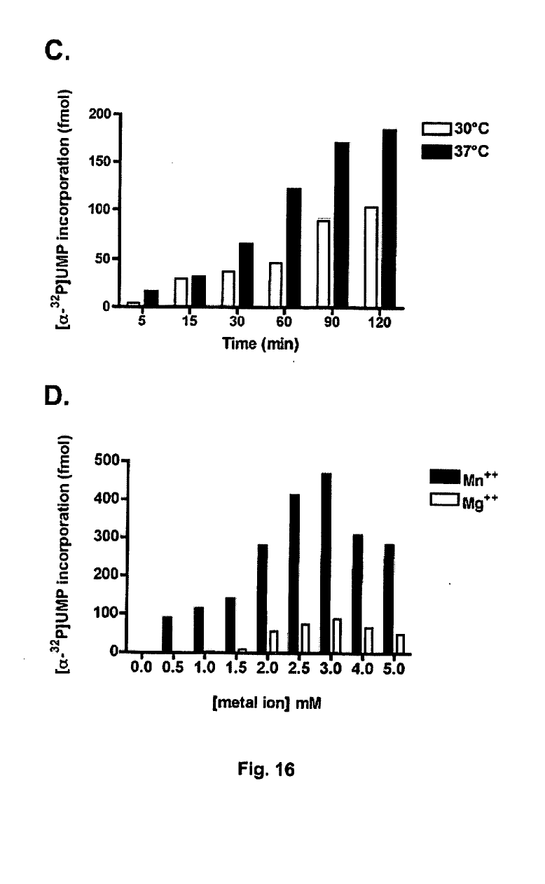

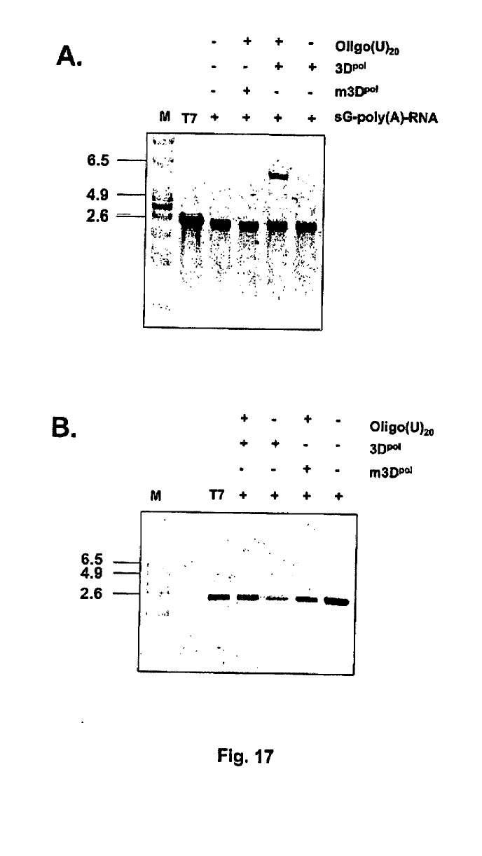

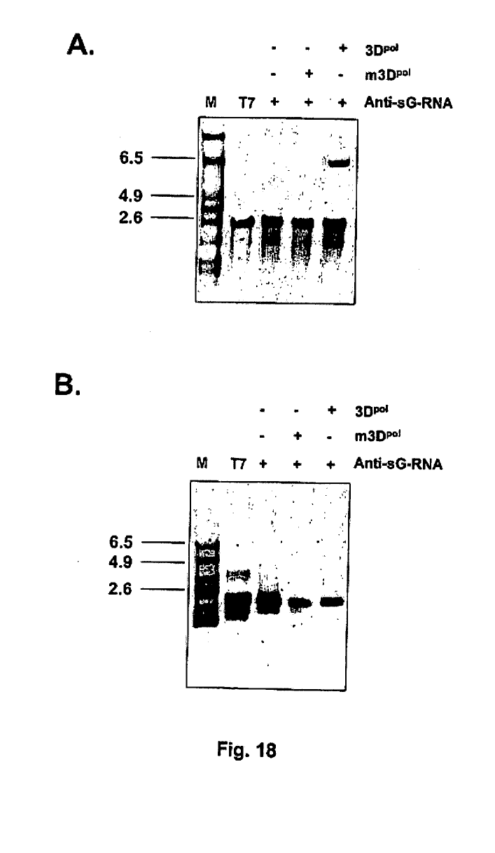

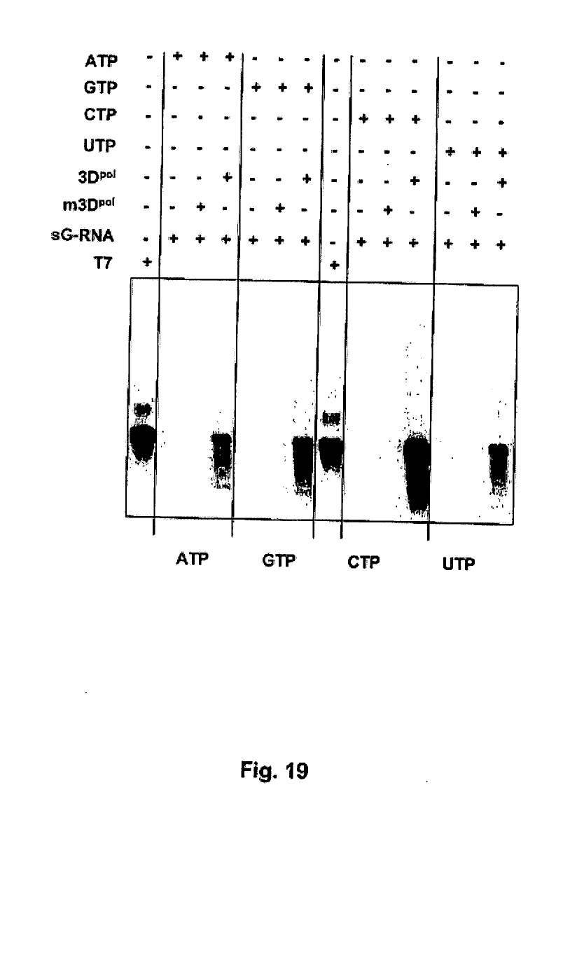

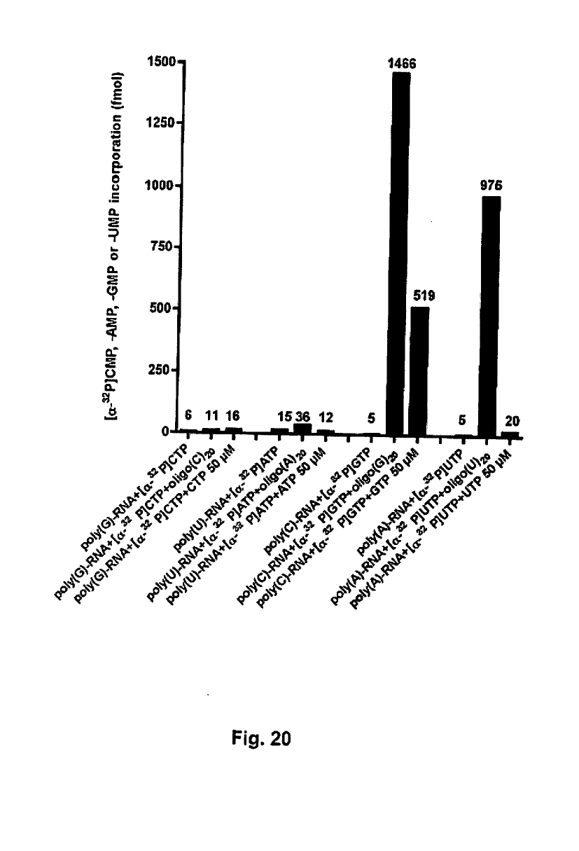

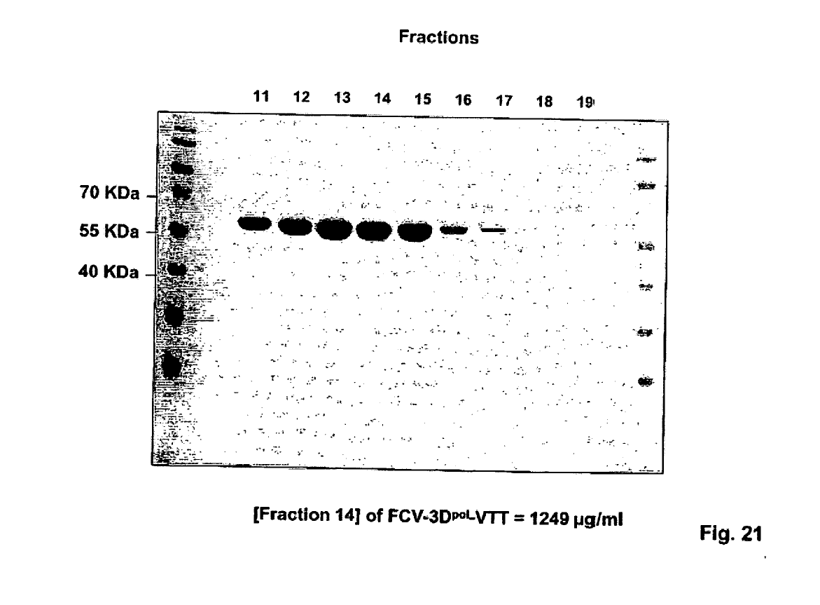

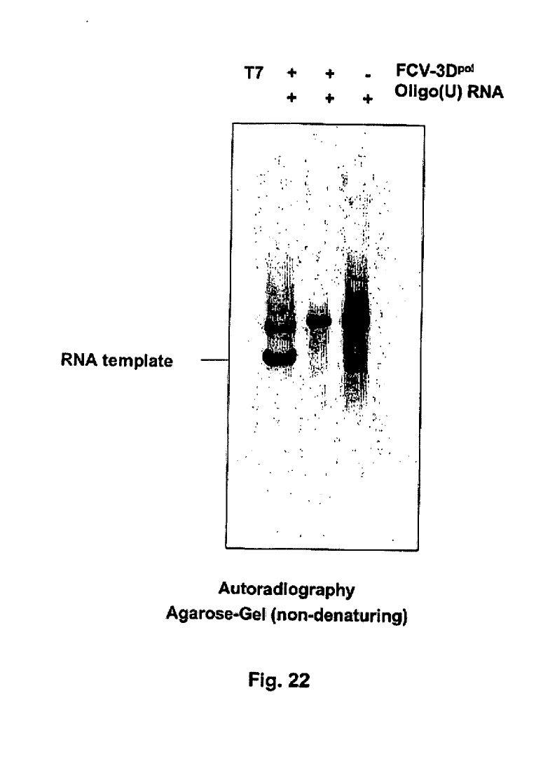

FIG. 5: expression and purification of norovirus 3D^{pol} in E. coli.
(A) SDS-PAGE analysis of purified recombinant norovirus 3D^{pol} having a C-terminal His-tag expressed in E. coli. 3D^{pol}, wild type norovirus 3D^{pol}, m3D^{pol}, norovirus 3D^{pol} having a mutation in the active site (YGD_{343G}D_{344G}). M, molecular weight marker (KDa).
(B) Western-blot analysis of purified recombinant norovirus 3D^{pol} using a pentahistidine antibody (monoclonal mouse antibody Qiagen) for the detection of proteins having a C-terminal His-tag. 3D^{pol}, wild type norovirus 3D^{pol}. Norovirus 3D^{pol} having a mutation in the active site (YGD_{343G}D_{344G}). M, molecular weight marker (KDa).

FIG. 6: RNA synthesis with norovirus 3D^{pol}.
(A) The RNA synthesis was examined as indicated in the presence of a synthetic subgenomic RNA (sG-RNA) as template as well as in the presence of wild type norovirus 3D^{pol} or a norovirus 3D^{pol} having a mutation in the active site (YGD_{343G}D_{344G}). The reaction products were analysed on a non-denaturating agarose gel and visualized by using autoradiography. T7, synthetic subgenomic RNA produced by in vitro transcription using T7.
(B) Ethidium bromide staining of the same gel and visualisation of the reaction products through UV transillumination. The visible band remaining in the case of the reaction with m3D^{pol} instead of wild type 3D^{pol} stems from the synthetic subgenomic RNA template used in the reaction. M, RNA molecular weight marker.
(C) Strand separation analysis of the reaction product of the in vitro RNA synthesis using norovirus 3D^{pol}. The reaction product was produced by RNA synthesis using norovirus 3D^{pol} (3D^{pol}) using a synthetic subgenomic RNA (sG-RNA) as template. The reaction products were visualised on non-denaturating (0.5 M; left hand side) and denaturating (1.25 M; right hand side) formaldehyde agorose gels. T7, synthetic subgenomic RNA produced by means of in vitro transcription using T7. M, RNA molecular weight marker.

FIG. 7: Northern blot analysis of the products of norovirus 3D^{pol} synthesis.
(A) The RNA synthesis was carried out as indicated in the presence of a synthetic subgenomic RNA (sG-RNA) as template as well as in the presence of wild type norovirus 3D^{pol} or a norovirus 3D^{pol} having a mutation in the active site (YGD_{343G}D_{344G}). The reaction products were analysed on a non-denaturating agorose gel and visualised by means of UV transillumination after ethidium bromide staining. T7, synthetic subgenomic RNA prepared by means of in vitro transcription using T7. M, RNA molecular weight marker. The visible band remaining in the case of the reaction with m3D^{pol} instead of wild type 3D^{pol} sterns from the template used in the reaction.
(B) Hybridisation of a (−)-stranded RNA probe with the products of the synthesis of the norovirus 3D^{pol}. T7, synthetic subgenomic RNA produced by means of in vitro transcription using T7. sG-RNA, synthetic subgenomic RNA used as template. 3D^{pol}, wild type norovirus 3D^{pol}. Norovirus 3D^{pol} having a mutation in the active site (YGD_{343G}D_{344G}).

FIG. 8 Analysis of the concentration dependency, the temperature dependency and the variation in time of the norovirus 3D^{pol} activity. Subgenomic RNA was used as template in all reactions.
(A) Concentration dependent activity of the norovirus 3D^{pol}. Norovirus 3D^{pol} concentration (μM) as indicated. The reaction was carried out for 2 h at 30° C. Mean value and standard deviation of three independent experiments are shown for each concentration.
(B) Variation-in-time analysis of the norovirus 3D^{pol} activity. Incorporation of [α-³²P]UMP as indicated. The reaction was carried out at 30° C., and 3 μM norovirus 3D^{pol} were used. Mean value and standard deviation of three independent experiments are shown for each time point.
(C) Temperature dependent activity of the norovirus 3D^{pol}. Incorporation of
   [α-³²P]UMP as indicated. The reaction time was 2 h. Mean value and standard deviation of three independent experiments are shown for each concentration.
(D) Metal ion dependency of the norovirus 3D^{pol} activity. RNA synthesis was carried out in the presence of 3 μM norovirus 3D^{pol}, subgenomic RNA as template (0.024 μM) and increasing concentrations (0.5 to 3 mM) of MgAcO, MnCl₂ or FeSO₄ for 2 h at 30° C. Incorporation of [α-³²P]UMP as indicated. Mean value and standard deviation of three independent experiments are shown for each concentration.

FIG. 9 Primer-dependent initiation of the RNA synthesis on homopolymeric templates.
RNA synthesis was carried out in the presence (black bars) or in the absence (grey bars) of a complementary RNA oligonucleotide primer. The incorporation of [α-³²P]CMP, [α-³²P]AMP, [α-³²P]GMP or [α-³²P]UMP was measured after TCA precipitation and collection from G/C glass fibre filters. The incorporation values are indicated.

FIG. 10: Terminal transferase activity of the norovirus 3D^{pol}
(A) In order to examine the terminal transferase activity, 3 μM norovirus 3D^{pol} were incubated for 2 h at 30° C. in the presence of subgenomic RNA (sG-RNA) as template (0.024 μM) and [α-³²P]ATP, [α-³²P]GTP, [α-³²P]CTP or [α-³²P]UTP as indicated. The reaction products were analysed on non-denaturating agarose gels and visualised by autoradiography.
(B) Variation-in-time experiment of the norovirus 3D^{pol} transferase activity on subgenomic norovirus RNA as template. The reaction was carried out as described in (A) but using [α-³²P]UTP as labelled nucleotide, stopped at the indicated time points (sec), and the reaction products were visualised on non-denaturating agarose gels by means of autoradiography. T7, synthetic subgenomic RNA produced by in vitro transcription using T7.
(C) Variation-in-time experiments of the RNA synthesis with 3 μM norovirus 3D^{pol} by using subgenomic RNA as a template. The reaction was incubated at 30° C., stopped at the indicated time points (sec), and the reaction products were visualised on non-denaturation agarose gels by means of audio radiography. T7, synthetic subgenomic RNA produced in vitro transcription using T7. P, product of norovirus 3D^{pol} synthesis.

FIG. 11: Primer-dependent replication of full-length subgenomic polyadeylated by norovirus 3D^{pol}. Synthetic subgenomic polyadenylated RNA was used as template in all reactions. The reaction products were analysed on formaldehyde agarose gels and visualised through autoradiography.
(A) The reaction was carried out in the presence of an oligo(U)₂₀ RNA primer as indicated (mM). M, marker (in vitro transcribed subgenomic polyadenylated RNA).
(B) The reaction was carried out in the presence of an RNA oligonucleotide which is complementary to the sequence adjacent to the poly(A) tail. RNA oligonucleotide primer concentration (mM) as indicated. M, marker (in vitro transcribed subgenomic polyadenylated RNA).

FIG. 12: De novo initiation of RNA synthesis on norovirus anti-subgenomic RNA.
Synthetic anti-subgenomic RNA was used as template for the replication in all reactions. The RNA reaction products were analysed on native agarose gels and visualised through UV transillumination after ethidium bromide staining.
(A) Lanes 1 to 3: RNA synthesis in the presence of wild type 3D^{pol}, mutated 3D^{pol} and in the absence of 3D^{pol}, respectively. Lanes 4 to 6: RNA synthesis in the presence of wild type 3D^{pol} and an RNA oligonucleotide complementary to the 3′-terminus, mutated 3D^{pol} and an RNA oligonucleotide complementary to the 3′-terminus and with an RNA oligonucleotide complementary to the 3′-terminus but without norovirus 3D^{pol}, respectively. Lanes 7 to 9, RNA synthesis in the presence of wild type 3D^{pol} and a DNA oligonucleotide complementary to the 3′-terminus, mutated 3D^{pol} and a DNA oligonucleotide complementary to the 3′-terminus and with a DNA oligonucleotide complementary to the 3′-terminus but without norovirus 3D^{pol}, respectively. T, template RNA. R, replication product. M, RNA molecular weight marker (Kb).
(B) Strand separation analysis of the replication products. The reaction products were analysed on formaldehyde agarose gels and visualised through UV transillumination after ethidium bromide staining. Lanes 1 and 2, template (anti-subgenomic RNA) and norovirus 3D^{pol} replication product, respectively. M, RNA molecular weight marker (Kb).

FIG. 13: Primer-independent de novo initiation of RNA synthesis on homopolymeric templates. The RNA synthesis was carried out in the presence (black bars) or in the absence (grey bars) of cold CTP, ATP, GTP and UTP, respectively, for poly(rG), poly(rU), poly(rC) and poly(rA), respectively, templates. The integration of [α-³²P]CMP, [α-³²P]AMP, [α-³²P]GMP or [α-³²P]UMP was measured after TCA precipitation and collecting from G/C glass fibre filters. The incorporation values are indicated.

FIG. 14: Amplification of viral and eukaryotic RNA using the norovirus RNA polymerase enzyme.
(A) Autoradiograph showing the incorporation of [α-³²P]UMP. Reaction 1, subgenomic norovirus RNA; reaction 2, subgenomic norovirus RNA having a 60 nucleotide deletion at the 5′-end of the RNA; reaction 3, subgenomic norovirus RNA having a 60 nucleotide deletion at the 3′-end of the RNA; reaction 4, subgenomic norovirus RNA having a 60 nucleotide deletion at the 5′-end and 3′-end of the RNA; reaction 5 and 6, negative control; reaction 7; subgenomic astrovirus RNA (serotype 1); reaction 8; subgenomic astrovirus RNA (serotype 2); reaction 9, eukaryotic RNA of the elF4 gene of Xenopus laevis; reaction 10, subgenomic norovirus DNA; reaction 11; T7-transcribed RNA of the elF4 gene of Xenopus laevis; reaction 12, negative control.
(B) Quantification of the incorporation of [α-³²P]UMP. The numbering of the reactions refers to the samples of FIG. 14 (A).

FIG. 15: Expression and purification of sapovirus 3D^{pol} in E. coli.
(A) SDS-PAGE analysis of purified recombinant sapovirus 3D^{pol} expressed in E. coli and carrying a C-terminal His-tag. 3D^{pol}, wild type sapovirus 3D^{pol}. m3D^{pol}, sapovirus 3D^{pol} having a mutation in the active site (YG_{343G}D_{344G}). M, molecular weight marker (KDa).
(B) Western blot analysis of purified recombinant sapovirus 3D^{pol} using a pentahistidine antibody (monoclonal mouse antibody, Qiagen) for the detection of proteins having a C-terminal His-tag. 3D^{pol}, wild type sapovirus 3D^{pol}. m3D^{pol}, sapovirus 3D^{pol} having a mutation in the active site (YGD_{343G}D_{344G}). M, molecular weight marker (KDa).

FIG. 16: Concentration dependency, substrate dependency, temperature dependency and metal ion dependency of the sapovirus 3D^{pol} activity. Subgenomic RNA was used as template in all reactions.
(A) Concentration-dependent activity of the sapovirus 3D^{pol}. RNA synthesis was carried out in the presence of 0.024 μM RNA and increasing concentrations of sapovirus 3D^{pol} (0.0 to 5.0 μM). Incorporation of [α-³²P]UMP as indicated. The reaction was carried out for 2 h at 37° C.
(B) Substrate dependency of the sapovirus 3D^{pol} activity. RNA synthesis was carried out in the presence of 3 μM sapovirus 3D^{pol} and increasing concentrations of subgenomic RNA as template (0.00 to 0.100 μM) for 2 h at 37° C. Incorporation of [α-³²P]UMP as indicated.
(C) Temperature-dependent activity of the sapovirus 3D^{pol}. The reaction was carried out at 30° C. or 37° C. and stopped at the indicated time points. 3 μM sapovirus 3D^{pol} was used in all reactions. Incorporation of [α-³²P]UMP as indicated.
(D) Metal ion dependency of the sapovirus 3D^{pol} activity. RNA synthesis was carried out in the presence of 3 μM sapovirus 3D^{pol} using subgenomic RNA as template (0.024 μM) and increasing concentrations (0.5 to 5 mM) MgAcO or MnCl₂ at 37° C. for 2 h. Incorporation of [α-³²P]UMP as indicated.

FIG. 17: RNA synthesis using sapovirus 3D^{pol}.
(A) RNA synthesis in the presence of synthetic subgenomic polyadenylated RNA (sG-poly(A)-RNA) as template was examined with or without addition of a 3 μM oligo(U)₂₀ RNA primer and in the presence of wild type sapovirus 3D^{pol} (3D^{pol}) or, as indicated, of a 3D^{pol} having a mutation in the active site (m3D^{pol}, YGD_{343G}D_{344G}). The reaction products were analysed on non-denaturating agarose gels and visualised using UV transillumination after ethidium bromide staining. The visible remaining band in those reactions carried out with m3D^{pol} instead of wild type 3D^{pol} or without addition of oligo(U)₂₀ RNA primer, resulted from the synthetic subgenomic RNA template used in the reaction. T7, synthetic subgenomic RNA prepared by means of in vitro transcription using T7. M, RNA molecular weight marker.
(B) Strand separation analysis of the reaction products of in vitro RNA synthesis using sapovirus 3D^{pol}. The reaction product was obtained as indicated by RNA synthesis with sapovirus 3D^{pol} (3D^{pol}) using a subgenomic polyadenylated RNA (sG-poly(A)-RNA) as template. The reaction products were visualised on denaturating formaldehyde agarose gels. T7, synthetic subgenomic RNA prepared by means of in vitro transcription using T7. M, RNA molecular weight marker.

FIG. 18: De novo initiation of RNA synthesis on ant-sugenomic sapovirus RNA.
(A) RNA synthesis was carried out in the presence of a synthetic anti-subgenomic RNA (anti-sG-RNA) as template and wild type sapovirus 3D^{pol} (3D^{pol}) or a 3D^{pol} mutated in the active site (m3D^{pol}, YGD_{343G}D_{344G}) as indicated. The reaction products were analysed on non-denaturating agarose gels and visualised through UV transillumination after ethidium bromide staining. The visible remaining band in the reaction in which m3D^{pol} instead of wild type 3D^{pol} was used results from the synthetic anti-subgenomic RNA template used in the reaction. T7, synthetic subgenomic RNA prepared by means of in vitro transcription using T7. M, RNA molecular weight marker.
(B) Strand separation analysis of the reaction product of the in vitro RNA synthesis using sapovirus 3D^{pol}. The reaction product was obtained by RNA synthesis with sapovirus 3D^{pol} using a synthetic anti-subgenomic RNA (anti-sG-RNA) as template as indicated. The reaction products were visualised on denaturating formaldehyde agarose gels. T7, synthetic anti-subgenomic RNA prepared by means of in vitro transcription using T7. M, RNA molecular weight marker.

FIG. 19: Terminal transferase activity of sapovirus 3D^{pol}.
The terminal transferase activity was examined on the basis of a reaction using 3 μM sapovirus 3D^{pol} or an active site-mutated 3D^{pol} (m3D^{pol}) incubated in the presence of subgenomic RNA (sG-RNA) as template (0.024 μM) and [α-³²P]ATP, [α-³²P]GTP, [α-³²P]CTP or [α-³²P]UTP for 2 h at 37° C. (0.024 μM) as indicated. The reaction products were analysed on non-denaturating agarose gels and visualised by autoradiography. T7, synthetic subgenomic RNA prepared by means of in vitro transcription using T7.

FIG. 20: De novo initiation of RNA synthesis by sapovirus 3D^{pol} on homopolymeric templates.
RNA synthesis was performed by incubating poly(G)-RNA, poly(U)-RNA, poly(C)-RNA and poly(A)-RNA templates with [α-³²P]CTP, [α-³²P]ATP, [α-³²P]GTP and [α-³²P]UTP, respectively, and with or without 3 μM of an oligo(C)₂₀, oligo(A)₂₀, oligo(G)₂₀ or oligo(U)₂₀ RNA primer, respectively. In parallel, the RNA synthesis was performed in the absence of an oligo(RNA)-primer but in the presence of 50 μM cold CTP, ATP, GTP and UTP, respectively, for poly(G)-RNA, poly(U)-RNA, poly(C)-RNA or poly(A)-RNA templates, respectively. Incorporation of [α-³²P]CMP, [α-³²P]AMP, [α-³²P]GMP or [α-³²P]UMP as indicated.

FIG. 21: Expression and purification of vesivirus 3D^{pol} in E. coli.
(A) SDS-PAGE analysis of purified recombinant vesivirus 3D^{pol} having a C-terminal His-tag expressed in E. coli. Fractions 11 to 19 are shown after affinity purification on a Ni-NTA-column as are the concentrations of fraction 14. M, molecular weight marker (KDa).

FIG. 22: RNA synthesis using vesivirus 3D^{pol}.
(A) RNA synthesis was examined in the presence of a synthetic subgenomic polyadenylated RNA (sG-poly(A)-RNA) as template and with and without addition of a 3 μM oligo(U)₂₀ RNA primer, and in the presence of wild type vesivirus 3D^{pol} (3D^{pol}). The reaction products were analysed on non-denaturation agarose gels and visualised after autoradiography. T7, synthetic subgenomic RNA prepared by in vitro transcription using T7. M, RNA molecular weight marker.

### DETAILED DESCRIPTION OF THE INVENTION

### Example 1

### Method for the production of recombinant norovirus RdRP

The cDNA of the norovirus RdRP was obtained by PCR from norovirus clone pUS-NorII (GenBank accession number: AY741811). It was cloned into the pET-28b(+) vector (Novagen), the expression vector was sequenced and transformed into E. coli CL21 (DE3) pLysS. Cells were cultured at 37° C. in Luria-Bertani medium with kanamycin (50 mg/l). The protein expression was induced at an optical density of 0.6 (OD600) by the addition of isopropyl-β-D-thiogalactopyranoside (IPTG) to a final concentration of 1 mM. Cultures were then incubated at 25° C. over night. Cell pellets obtained from a 250 ml culture were washed once in 4 ml phosphate-buffered saline (PBS) and 1% Triton X 100 (sigma). Cells were treated at 37° C. for 15 minutes with DNase (10 U/ml), sonified on ice and resuspended in 40 ml binding buffer (20 mM Tris/HCl, pH 7.9, 500 mM NaCl, 5 mM imidazole). The cleared lysate was obtained upon centrifugation at 4300 rpm at 4° C. for 40 minutes. The norovirus RdRP provided with a His6-tag was bonded to a Ni-nitrilotriacetic acid (NTA) sepharose matrix (Novagen) which had been preequilibrated with binding buffer. The bound protein was washed with binding buffer containing 60 mM imidazole and eluted with binding buffer containing 1 M imidazole. The eluted protein was dialysed against buffer A (25 mM Tris-HCl, pH 8.0, 1 mM β-mercaptoethanol, 100 mM NaCl, 5 mM MgCL₂, 10% glycerine, 0.1% Triton X). The protein concentration was determined using the BCA protein assay kit (Pierce) based on the Biuret reaction. The enzyme was resuspended in a final volume in 50% clycerine and stored at −20° C.

### Example 2

### Sequence-Independent RNA Amplification Using Norovirus-RdRP

The reaction mixture (50 μl) consists of 0.5 to 1 μg RNA template, 10 μl reaction buffer (250 mM HEPES, pH 8.0, 15 mM magnesium acetate, 20 mM DTT), 50 U RNase inhibitor (RNAsin, Promega), 0.4 mM of each of ATP, CTP, GTP, UTP, 3 μM norovirus-RdRP prepared according to Example 1. The reaction is carried out at 30° C. for 2 h. The reaction is stopped by adding 50 μl stop solution (4 M ammonium acetate, 100 mM EDTA). The purification is performed by phenol/chloroform extraction or by means of the MEGAclear kit (Ambion) according to the manufacturer's instructions. The transcription products are made visible through UV transillumination on TBE-buffered agarose gels after ethidium bromide staining. Formamide agarose gels can be used as well.

### Example 3

### Sequence-Dependent RNA Amplification with Norovirus-RdRP Using a Gene Specific RNA Primer

The reaction mixture (50 μl) consists of 0.5 to 1 μg RNA template, 10 μl reaction buffer (250 mM HEPES, pH 8.0, 15 mM magnesium acetate, 20 mM DTT), 50 U RNase inhibitor (RNAsin, Promega), 0.4 mM of each of ATP, CTP, GTP, UTP, 0.1 to 1 μM gene specific RNA primer, 3 μM norovirus-RdRP prepared according to Example 1. The reaction is performed at 30° C. for 2 h. The reaction is stopped by adding 50 μl stop solution (4 M ammonium acetate, 100 mM EDTA). The purification is carried out by phenol/chloroform extraction or by means of the MEGAclear kit (Ambion) according to the manufacturer's instructions. The transcription products are made visible by UV transillumination on TBE-buffered agarose gels after ethidium bromide staining. Formaldehyde agarose gels or urea/polyacrylamide gels can also be used.

### Example 4

### Amplification of the Cellular mRNA with Norovirus-RdRP Using a Poly-U-Primer

The reaction mixture (50 μl) consists of 0.5 to 1 μg RNA template, 10 μl reaction buffer (250 mM HEPES, pH 8.0, 15 mM magnesium acetate, 20 mM DTT), 50 U RNase inhibitor (RNAsin, Promega), 0.4 mM of each of ATP, CTP, GTP, UTP, 0.1 to 1 μM poly-(U)₂₀-primer, 3 μM norovirus-RdRP prepared according to Example 1. The reaction is performed at 30° C. for 2 h. The reaction is stopped by adding 50 μl stop solution (4 M ammonium acetate, 100 mM EDTA). The purification is carried out by phenol/chloroform extraction or by means of the MEGAclear kit (Ambion) according to the manufacturer's instructions. The transcription products are made visible by UV transillumination on TBE-buffered agarose gels after ethidium bromide staining. Formaldehyde agarose gels can also be used.

### Example 5

### Production of siRNA Using Norovirus RdRP

The reaction mixture (50 μl) consists of 0.5 to 1 μg RNA template, 10 μl reaction buffer (250 mM HEPES, pH 8.0, 15 mM magnesium acetate, 20 mM DTT), 50 U RNase inhibitor (RNAsin, Promega), 0.4 mM of each of ATP, CTP, GTP, UTP, 3 μM norovirus-RdRP prepared according to Example 1. The reaction is performed at 30° C. for 2 h. The reaction is stopped by adding 50 μl stop solution (4 M ammonium acetate, 100 mM EDTA). The purification is carried out by phenol/chloroform extraction or by means of the MEGAclear kit (Ambion) according to the manufacturer's instructions. The transcription products are made visible by UV transillumination on TBE-buffered 10% polyacrylamide gels after ethidium bromide staining. Formaldehyde agarose gels can also be used. Alternatively, armored-RNA can be used.

### Example 6

### Labelling of RNA Using Norovirus RdRP

The reaction mixture (50 μl) consists of 0.5 to 1 μg RNA template, 10 μl reaction buffer (250 mM HEPES, pH 8.0, 15 mM magnesium acetate, 20 mM DTT), 50 U RNase inhibitor (RNAsin, Promega), 0.4 mM of ATP or CTP or GTP or UTP, 3 μM norovirus-RdRP prepared according to Example 1. The reaction is performed at 30° C. for 2 h. The reaction is stopped by adding 50 μl stop solution (4 M ammonium acetate, 100 mM EDTA). The purification is carried out by phenol/chloroform extraction or by means of the MEGAclear kit (Ambion) according to the manufacturer's instructions. The transcription products are made visible by UV transillumination on TBE-buffered 10% polyacrylamide gels after ethidium bromide staining. Formaldehyde agarose gels can also be used. Alternatively, armored-RNA can be used.

### Example 7

### RNA Synthesis of Poly(C)-Poly(G) RNA Hybrid Using Norovirus-RdRP

The reaction mixture (50 μl) consists of 0.5 to 1 μg RNA template, 10 μl reaction buffer (250 mM HEPES, pH 8.0, 15 mM magnesium acetate, 20 mM DTT), 50 U RNase inhibitor (RNAsin, Promega), 50 μM GTP, and 3 μM norovirus-RdRP prepared according to Example 1. The reaction is carried out at 30° C. for 2 h. The reaction is stopped by adding 50 μl stop solution (4 M ammonium acetate, 100 mM EDTA). The purification is performed phenol/chloroform extraction or by means of the MEGAclear kit (Ambion) according to the manufacturer's instructions. The transcription products are made visible by UV transillumination on TBE-buffered 10% polyacrylamide gels after ethidium bromide staining. Formaldehyde agarose gels can also be used. Alternatively, armored-RNA can be used.

What is claimed is:

1. A RNA-dependent RNA polymerase having a right hand conformation and an amino acid sequence comprising the following sequence motifs:
XXDYS
GXPSG
YGDD
XXYGL
XXXXFLXRXX
wherein the amino acids in the sequence motifs are identified as:
D: aspartate
Y: tyrosine
S: serine
G: glycine
P: proline
L: leucine
F: phenylalanine
R: arginine
X: any amino acid.

2. The RNA-dependent RNA polymerase of claim 1 wherein the RNA-dependent RNA polymerase is an RNA-dependent RNA polymerase of a virus of the Caliciviridae family.

3. The RNA-dependent RNA polymerase of claim 2 wherein the RNA-dependent RNA polymerase is an RNA-dependent RNA polymerase of a human and/or non-human pathogenic virus of the Caliciviridae family.

4. The RNA-dependent RNA polymerase of claim 3 wherein the RNA-dependent RNA polymerase is an RNA-dependent RNA polymerase of a noroviurs, sapovirus, vesivirus or lagovirus.

5. The RNA-dependent RNA polymerase of claim 4 wherein the RNA-dependent RNA polymerase is a RNA-dependent RNA polymerase of: norovirus strain HuCV/NL/Dresden 174/1997/GE, or sapovirus strain pJG-Sap01, or vesivirus strain FCV/Dresden/2006/GE.

6. The RNA-dependent RNA polymerase of claim 4 wherein the RNA-dependent RNA polymerase is a protein according to SEQ ID NO 1, SEQ ID NO 2, SEQ ID NO 3, SEQ ID NO 4, SEQ ID NO 5 or SEQ ID NO 6.

7. A method for the amplification of RNA using an RNA-dependent RNA polymerase having a right hand conformation and an amino acid sequence comprising the following sequence motifs:
XXDYS
GXPSG
YGDD
XXYGL
XXXXFLXRXX
wherein the amino acids in the sequence motifs are identified as:
D: aspartate
Y: tyrosine
S: serine
G: glycine
P: proline
L: leucine
F: phenylalanine
R: arginine
X: any amino acid
comprising the steps of:
a. annealing of the RNA-dependent RNA polymerase to the RNA template in the presence or absence of an oligonucleotide primer;
b. transcribing the RNA template into antisense-RNA by the RNA-dependent RNA polymerase;
c. separating the RNA/antisense-RNA duplex into individual strands.

8. The method for the amplification of RNA of claim 7 characterized in that the separation of the RNA/antisense-RNA duplex into individual strands occurs by heat denaturation, chemical denaturation and/or enzymatically.

9. The method for the amplification of RNA of claim 8 characterized in that the enzymatic separation of the RNA/antisense-RNA duplex into individual RNA strands is carried out by an enzyme capable of separating double-stranded RNA into single-stranded RNA.

10. The method for the amplification of RNA of claim 7 wherein at least one primer hybridizing with a section of the RNA template is used and that the primer is elongated by the RNA-dependent RNA polymerase according to the sequence of the RNA template after annealing of the RNA-dependent RNA polymerase.

11. The method for the amplification of RNA of claim 10 characterized in that a heteropolymeric or homopolymeric RNA primer or DNA primer is used as the primer.

12. The method for the amplification of RNA of claim 11 characterized in that the primer is a poly-U-RNA, poly-A-RNA, poly-C-RNA or poly-G-RNA primer or any homopolymeric oligo-RNA primer.

13. The method for the amplification of RNA of claim 7 wherein no primer hybridizing with a section of the RNA template is used and elongated by the RNA-dependent RNA polymerase according to the sequence of the RNA template after annealing of the RNA-dependent RNA polymerase.

14. The method for the amplification of RNA of claim 7, wherein said RNA comprises poly(C)-RNA, and no primer hybridizing with a section of the RNA template is used, and GTP is used as a single nucleotide and elongated by the RNA-dependent RNA polymerase according to the sequence of the RNA template.

15. A method for the labelling of RNA using RNA-dependent RNA polymerase having a right hand conformation and an amino acid sequence comprising the following sequence motifs:
XXDYS
GXPSG
YGDD
XXYGL
XXXXFLXRXX
wherein the amino acids in the sequence motifs are identified as:
D: aspartate
Y: tyrosine
S: serine
G: glycine
P: proline
L: leucine
F: phenylalanine
R: arginine
X: any amino acid;
comprising the steps of:
a. annealing the RNA-dependent RNA polymerase to the RNA to be labelled.
b. adding at least one nucleotide to the 3′-end of the RNA to be labelled.

16. A kit for the amplification of RNA comprising:
an RNA-dependent RNA polymerase having a right hand conformation and an amino acid sequence comprising the following sequence motifs:
XXDYS
GXPSG
YGDD
XXYGL
XXXXFLXRXX
wherein the amino acids in the sequence motifs are identified as:
D: aspartate
Y: tyrosine
S: serine
G: glycine
P: proline
L: leucine
F: phenylalanine
R: arginine
X: any amino acid;
a reaction buffer;
NTPs;
optionally, RNase inhibitor;
optionally, stop solution.

17. A kit for labelling RNA comprising:
an RNA-dependent RNA polymerase having a right hand conformation and an amino acid sequence comprising the following sequence motifs:
XXDYS
GXPSG
YGDD
XXYGL
XXXXFLXRXX
wherein the amino acids in the sequence motifs are identified as:
D: aspartate
Y: tyrosine
S: serine
G: glycine
P: proline
L: leucine
F: phenylalanine
R: arginine
X: any amino acid;
a reaction buffer;
NTPs;
optionally, RNase inhibitor;
optionally, stop solution.


SEQUENCE LISTING (6 sequences)

SEQ ID NO: 1 - protein, 520 aa, Norovirus
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALE

SEQ ID NO: 2 - protein, 517 aa, Sapovirus
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 3 - protein, 533 aa, Norovirus
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLE

SEQ ID NO: 4 - protein, 526 aa, Artificial
  Norovirus-RdRP having a histidine marker
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALEHHHHHH

SEQ ID NO: 5 - protein, 523 aa, Artificial
  Sapovirus-RdRP having a histidine marker
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEMEHHHHHH

SEQ ID NO: 6 - protein, 539 aa, Artificial
  Vesivirus-RdRP having a histidine marker
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLEHHHHHH